(19) United States
(12) Patent Application Publication
(10) Pub. No.: US 2009/0209973 A1
(43) Pub. Date: Aug. 20, 2009

(54) Surgical apparatus and the use thereof

(76) Inventor: Michael C. East, Christchurch (NZ)

Correspondence Address: JACOBSON HOLMAN PLLC, 400 SEVENTH STREET N.W., SUITE 600, WASHINGTON, DC, 20004, US

(21) Appl. No.: 11/921,647

(22) PCT Filed: Jun. 8, 2006
(86) PCT No.: PCT/NZ2006/000143

§ 371 Date: Feb. 26, 2008

(30) Foreign Application Priority Data
Jun. 10, 2005 (NZ) 540710

(51) Int. Cl. A61B 17/42

(52) U.S. Cl. 606/119

(57) ABSTRACT

Surgical apparatus for laparoscopic assisted vaginal hysterectomy. The apparatus has an elongate housing, having a handle end and a manipulator end; a diaphragm situated distal the handle end of the housing; a manipulator integral with or fitted in the manipulator end of the elongate housing; defining, the manipulator a tip extending distal of said housing and said handle end thereof, and a passage for passing gas from the handle end of the housing, to at least one exit in or adjacent to the manipulator and distal of the handle end beyond the diaphragm.

The manipulator tip can be manipulated sufficiently by the handle end despite the gas sealing presence of the diaphragm to enable manipulation of the uterus.

### TECHNICAL FIELD

This invention relates to surgical apparatus and the use thereof.

### BACKGROUND ART

Hysterectomies have traditionally been performed by making a cut through the abdomen wall, freeing up the uterus and removing the uterus through the cut in the wall. In more recent times some hysterectomies have been able to be performed through the vaginal opening which consists in freeing up the uterus by using laparoscopic instruments and then removing the uterus through an opening made at the top of the vagina.

Laparoscopic procedures require the abdomen to be inflated with gas, to form a pneumoperitoneum, which enables the surgeon to see what needs to be done and easily move the instruments around and position them to make the necessary cuts, sutures, etc. Thus laparoscopically assisted vaginal hysterectomy (LAVH) is considered an advanced laparoscopic operative procedure.

One difficulty with LAVH procedures is that once the cut is made at the top of the vagina in order to allow the uterus to be removed, the gas in the abdomen escapes, making it difficult and time consuming to finally release the uterus from the abdomen (cut ligaments, etc) and remove it through the vaginal opening. Therefore although LAVH allows patient to avoid undergoing abdominal hysterectomy procedures, thus affording them a more rapid recovery, the vaginal portion of the operation can still be extremely difficult if there is little descent of the cervix, especially when the uterus is very large. In such circumstances the vaginal portion of the surgery can take almost as long as the laparoscopic portion and can cause great stress to the surgeon and lead to significant blood loss. The more dissection performed laparoscopically, the easier the vaginal component of the surgery. Therefore, the longer pneumoperitoneum can be maintained the more efficient the procedure becomes. Bleeding and/or damage to the uterer is also a significant problem. Formation of the “bladder flap” can be difficult and hazardous with perforation of the bladder on occasions occurring. However, the bladder has to be dissected off the lower segment of the uterus before the uterine arteries can be approached In an attempt to make the “bladder flap” dissection easier, the initial dissection has been performed vaginally without opening the peritoneal fold of the utero-vesical space. However, if the fold of the peritoneum is breached, then gas can escape from the abdomen into the vagina thus deflating the abdomen and making it impossible to continue the surgery. In order to minimise this problem saline packs have been placed into the vagina in an attempt to slow down gas leak, but rarely does this prove to be satisfactory.

In addition to this, in order for the hysterectomy procedure to be completed efficiently via the laparoscope, it is necessary to manipulate certain organs to obtain access to make the necessary cuts, sutures etc. For example, it may be necessary to push the uterus high up into the abdomen to free the ligaments for access by the surgeon.

A further difficulty occurs in forming the pneumoperitoneum as standard techniques usually involve the blind insertion of the gas used via a Verres needle. As this is blind insertion of the gas, the operator cannot have complete confidence in where the gas is being inserted.

To this end several types of device has been proposed as a means of overcoming these difficulties.

U.S. Pat. No. 6,423,075; EP 0642766 A2 and Sabella et al in Obstet. Gynecol. 1996:87:465 proposed devices consisting of a handle, with a balloon cuff and manipulator at the distal end. By inflating the balloon the vagina can be occluded preventing the egress of gas thus allowing pneumoperitoneum to be maintained However none of these devices provide means for producing pneumoperitoneum thus not overcoming the difficulty of the operator having to carry out blind insertion of the gas as previously described.

Similarly U.S. Pat. No. 3,882,852 and U.S. Pat. No. 6,174,317 proposed devices with balloon cuffs. However these devices do not include uterine manipulators or a means of inducing pneumoperitoneum. Thus these devices do not allow any of the previously discussed difficulties to be overcome.

Similarly U.S. Pat. No. 6,572,631 proposes a device to occludes the vagina using a seal structure. The device allows pneumoperitoneum to be maintained however like the previous devices has no facility for introducing gas to create pneumoperitoneum.

U.S. Pat. No. 5,704,372 proposed a device features an inflatable balloon cuff and a duct intended for producing pneumoperitoneum. However this device was not intended for vaginal use and hence no provision was made for a uterine manipulator. Hence this device offers no advantage when performing LAVH procedures.

U.S. Pat. No. 5,643,285; U.S. Pat. No. 5,487,377 and U.S. Pat. No. 5,643,311 proposed inflatable cuff devices feature complex articulating, rotating or displaceable manipulators which add cost and complexity to the apparatus with no discernable gain in functionality. However none of these devices provide means for producing pneumoperitoneum thus not overcoming the difficulty of the operator having to carry out blind insertion of the gas as previously described.

U.S. Pat. No. 6,235,037 proposed an inflatable cuff device featuring a rotating or displaceable manipulator which could be replaced. This device differed from those above in that it provided a means of producing pneumnoperitoneum by passing gas through the handle of the device. However the inclusion of the rotating and displaceable manipulator adds cost and complexity to the apparatus with no discernable gain in functionality. The proposed device made no allowance for a fixed manipulator.

U.S. Pat. No. 5,431,662; U.S. Pat. No. 5,338,297; U.S. Pat. No. 6,802,825 and U.S. Pat. No. 5,935,098 proposed devices which provide occlusion of the uterus and manipulation of the uterus. However as these devices provide no means of achieving vaginal occlusion, pneumoperitoneum of the abdomen cannot be produced and thus these devices do not help overcome the previously detailed difficulties encountered when performing LAVH procedures.

The majority of the prior art discussed also utilises inflatable cuff devices to occlude the vaginal. The key deficiencies of inflatable cuff occluding devices are cost and complexity. In China it is estimated that 2.8 million hysterectomies are performed per year, and an estimated 2.3 million hysterectomies are performed per year in India However in these countries cost is a principle factor in determining uptake and utilisation of a device. Therefore given the costs associated with inflatable cuff devices, these devices are unlikely to be used and thus treatment practices will revert to abdominal hysterectomy techniques which have a less favorable patient outcome.

There is therefore a need for a low cost device capable of maintaining a pneumoperitoneum throughout LAVH procedures, this device should provide means to manipulate the uterus as may be necessary during the LAVH procedure. The device should also have provision for improving the creation of a pneumoperitoneum during LAVH procedures.

In some situations the operator may choose to produce or maintain pneumoperitoneum by means other than that provided for by the present invention. Accordingly the device should include a means to prevent the gas escaping via the mechanism to create pneumoperitoneum.

The field of surgery is also beginning to utilise mechanical means, such as robotic manipulation, for assisting surgeons in performing surgical procedures. This technology involves the surgeon directing the robotic mechanical manipulation means during the performance of a surgical procedure. In order for hysterectomies and other uterine procedures to be able to utilise this technology, it will be advantageous if not essential to provide a fixed fulcrum point about which and from which the manipulation device can be moved.

### DISCLOSURE OF INVENTION

The invention consists in a surgical apparatus comprising or including:

(a) an elongate housing, having a handle end and a manipulator end;

(b) a diaphragm situated distal the handle end of the housing,

(c) a manipulator integral with or fitted in the manipulator end of the elongate housing; defining, the manipulator a tip extending distal of said housing and said handle end thereof, and

(d) a passage for passing gas from the handle end of the housing, to at least one exit in or adjacent to the manipulator and distal of the handle end beyond the diaphragm,

wherein the diaphragm can be positioned in the vaginal tract and either without further deployment or with further deployment (i.e. inflation or other activation) establish a sufficient seal to define a gas inflatable peritoneal and abdominal cavity in which a pneumoperitoneum can be established by the passage of gas and its egress from said exit,

and therein the manipulator tip can be manipulated sufficiently by the handle end despite the gas sealing presence of the diaphragm to enable manipulation of the uterus.

Preferred manipulation is either or both manipulation of the uterus from within the uterus prior to the vaginal cut and/or the manipulation of the uterus from outside the uterus after the vaginal cut.

In one option the diaphragm comprises a flat ring of fixed volume.

In another option the diaphragm comprises a ring of fixed volume, the majority of which, or all of which is, dished towards the distal end of the apparatus.

In still another option the diaphragm comprises a ring of fixed volume, the majority of which, or all of which is, dished away from the distal end of the apparatus.

In still another option the diaphragm may be mechanically deployed from a collapsed or relaxed state to an expanded less relaxed state.

In yet a further option the diaphragm may be thermally deployed, from a collapsed state to an expanded state.

In yet another option the diaphragm may be self deploying from a collapsed state to an expanded state.

Preferably the passage for passing gas to the distal end of the device has multiple exit points.

In a further aspect the invention consists in a method of laparoscopic assisted vaginal hysterectomy comprising or including the steps of:
(i) inserting apparatus comprising or including

an elongate housing, having a handle end and a manipulator end;

a diaphragm situated distal the handle end of the housing;

a manipulator integral with or fitted in the manipulator end of the elongate housing, defining, the manipulator a tip extending distal of said housing and said handle end thereof and

a passage for passing gas from the handle end of the housing, to at least one exit in or adjacent to the manipulator and distal of the handle end beyond the diaphragm, into a vagina, and if necessary deploying the diaphragm, so as to have the diaphragm fix the apparatus in place and to provide a sealed peritoneal and abdominal cavity;
(ii) passing a gas through said passage and into the peritoneal and abdominal cavity to create pneumoperitoneum;
(iii) utilising the manipulator tip to manipulate the uterus from within the uterus prior to vaginal cut; and
(iv) utilising the manipulator tip to manipulate the uterus from outside the uterus after vaginal cut.

The manipulator tip preferably does not affect the vaginal cut in any way. The vaginal cut is made either before the device in introduced using standard surgical methods (non laparoscopic), or alternatively, the vaginal cut may be made after the device is in place and pneumoperitoneum has been established. In this latter case the vaginal cut would typically be made laparoscopically. However the instruments do not need access via the device.

In an aspect, the invention relates to a surgical apparatus comprising or including

an elongate housing, having a handle end and a manipulation end;

a diaphragm situated distal to the handle end of the housing which is adapted to expand and/or collapse and/or deform to occlude the vagina; and

a channel for passing a gas from the handle end of the apparatus, past the diaphragm, and having an exit in or adjacent to the manipulation end of the housing.

In still another aspect, the invention relates to a method of laparoscopically assisted vaginal hysterectomy, the method utilizing a surgical apparatus which includes a diaphragm adapted to expand and/or collapse and/or deform to occlude the vagina; the method comprising or including the steps of

inserting the apparatus with a diaphragm into a vagina;

utilising the diaphragm to occlude the vagina as required (i.e. of the apparatus) or a;

passing a gas through the gas channel and into a peritoneal and abdominal cavity to create a pneumoperitoneum;

utilizing the device to manipulate the uterus from within a uterus prior to the vaginal cut; and

utilizing the device to manipulate the uterus from outside the uterus after the vaginal cut.

The invention also comprises a surgical apparatus at least including:

a housing,

a diaphragm adapted to be substantially circumferentially around the housing,

manipulator tip which is connected to, or which forms an integral part of, the housing, and

a channel adapted to pass a gaseous substance.

Preferably a surgical apparatus has any one or more of:

an elongated housing,

a diaphragm capable of being expanded, collapsed or deformed to occlude the vagina,

a manipulator tip which is connected to, or which forms an integral part of, the housing, and

a channel adapted to pass a gaseous substance.

Preferably the diaphragm comprises a flat ring of fixed volume.

Preferably the gas channel extends along or through the housing and has an exit between the manipulation tip of the housing and the diaphragm.

In alternative configuration the gas channel may extend along or through the housing and have multiple exits between the manipulation tip and the diaphragm.

Preferably the gaseous substance is carbon dioxide.

The invention further comprises a method of laparoscopically assisted vaginal hysterectomy utilising a surgical apparatus which includes a diaphragm adapted to expand and/or collapse and/or deform as required to occlude the vagina, and manipulation means in the form of a tip extending from the housing;

the method comprising or including the steps of

inserting the apparatus with the diaphragm into the vagina,

expanding and/or collapsing and/or deforming the diaphragm as required to fix the apparatus in position and to seal the vagina,

utilising the manipulation tip to manipulate the uterus from within the uterus prior to the vaginal cut and to manipulate the uterus from outside the uterus after the vaginal cut

The method preferably involves inserting the apparatus into the vagina, expanding and/or collapsing and/or deforming the diaphragm as required to fix the apparatus in position and to seal the vagina, passing a gas through the or a gas channel (preferably the gas channel of the apparatus) and into the peritoneal and abdominal cavity to create a pneumoperitoneum,

utilising the manipulation tip to manipulate the uterus from within the uterus prior to the vaginal cut and to manipulate the uterus from outside the uterus after the vaginal cut.

Preferably the method includes use of a mechanical means to manipulate the surgical apparatus during the course of the vaginal hysterectomy.

The invention further comprises a surgical apparatus compromising.

(a) an elongated housing, having a handle end and a manipulator end;

(b) a diaphragm situated distal the handle end of the housing;

(c) a manipulator comprising a tip which extends from the manipulator end of the elongated housing; and

(d) a passage for passing gas from the handle end of the housing, to an exit in or adjacent to the manipulator end of the device.

Preferably the diaphragm comprises an expandable and/or collapsing and/or deforming device capable of occluding the vagina.

Preferably the diaphragm comprises a diaphragm adapted to be inflated and deflated substantially circumferentially from the elongated housing.

Preferably further comprising a passage extending from the handle end of the elongated housing to the diaphragm for inflating the diaphragm.

Preferably said passage includes means to prevent uncontrolled deflation of the diaphragm.

Preferably the diaphragm comprises a flat ring of fixed volume.

Preferably the diaphragm comprises a device of fixed dimensions which is sufficiently pliable that it is capable of deforming to occlude the vagina.

Preferably the diaphragm comprises a ring of fixed volume, the majority of which, or all of which is, dished towards the distal end of the apparatus.

Preferably the diaphragm may be thermally deployed from a collapsed state to an expanded state.

Preferably the diaphragm may be self deploying from a collapsed state to an expanded state.

Preferably the diaphragm may be manually deployed from a collapsed state to an expanded state and maintained and or controlled between either state.

Preferably the passage for passing gas to the distal end of the device has multiple exit points.

Preferably the passage for passing gas to the distal end of the device includes means to prevent egress of the gas used to create pneumoperitoneum.

The invention further comprises a method of laparoscopic assisted vaginal hysterectomy, the method utilising a surgical apparatus which includes a diaphragm, a gas channel, and a manipulator tip, the method comprising the steps of:

(a) inserting the apparatus into a vagina;

(b) where applicable deploying the diaphragm to fix the apparatus in place and to seal the vagina;

(c) passing a gas through a passage and into the peritoneal and adnominal cavity to create pneumnoperitoneum;

(d) utilising the handle end of the apparatus to alter the position of the manipulator tip to effect manipulation of the uterus from within the uterus prior to vaginal cut; and

(e) utilising the handle end of the apparatus to alter the position of the manipulator tip to effect manipulation of the uterus from outside the uterus after vaginal cut.

Preferably including manipulating the surgical apparatus by means of a robotic arm attached to the apparatus.

### BRIEF DESCRIPTION OF DRAWINGS

An embodiment of the present invention will now be described by way of example only with reference to the accompanying drawings in which:

FIG. 1 is a schematic representation of the medical instrument showing the instrument with a flat ring diaphragm, positioned in the vagina,

FIG. 1A is a diagram of the device of FIG. 1 showing preferred gas flows when deployed in use.

FIG. 2 is a schematic representation of the medical instrument showing the instrument with an inflatable diaphragm, positioned in a ballooning manner in the vagina,

FIG. 2A is a diagram of the device of FIG. 2 showing preferred gas flows when deployed in use.

FIG. 3 is a schematic representation of the medical instrument showing the instrument with a diaphragm dished towards the distal end of the instrument, positioned in the vagina,

FIG. 3A is a diagram of the device of FIG. 3 showing preferred gas flows when deployed in use.

FIG. 4 is a schematic representation of the medical instrument showing the instrument with a diaphragm dished away from the distal end of the instrument, positioned in the vagina,

FIG. 4A is a diagram of the device of FIG. 4 showing preferred gas flows when deployed in use.

FIG. 5 is a schematic representation of the medical instrument showing the instrument with a manually actuated mechanically deployed diaphragm, positioned in the vagina,

FIG. 5A is a diagram of the device of FIG. 5 showing preferred gas flows when deployed in use.

FIG. 6 is a schematic representation of the medical instrument showing the instrument with a thermally deployed diaphragm, positioned in the vagina,

FIG. 6A is a diagram of the device of FIG. 6 showing preferred gas flows when deployed in use.

FIG. 7 is a schematic representation of the medical instrument showing the instrument with a self deploying diaphragm, positioned in the vagina,

FIG. 7A is a diagram of the device of FIG. 7 showing preferred gas flows when deployed in use,

FIG. 8 is a diagrammatic view of a preferred device absent a pull on diaphragm or a fitted inflatable diaphragm but otherwise in accordance with the present invention in a perspective from one direction,

FIG. 9 is the device of FIG. 8 shown in a different perspective view, and

FIG. 10 is an exploded diagram of the device of FIGS. 8 and 9.

FIG. 1 depicts an embodiment of the medical apparatus for use in gynecological surgery and in particular laparoscopic assisted vaginal hysterectomy. The apparatus comprises of an elongated handle 5 with a handle end 4 and a manipulator end 6. Attached to or forming an integral part of the manipulator end is a manipulation tip 8 which is adapted to be inserted into the uterus 9. Situated at the manipulator end 6 of the apparatus is a deformable and/or expandable and/or collapsing flat diaphragm 11 which acts to occlude the vagina 7. Pneumoperitoneum can then be produced by passing gas from the handle 4 end of the device to the exit port 10 in the manipulator end 6 of the apparatus though a channel 3. In another embodiment the apparatus may also feature a restricting device 2 to prevent the transfer of gas through the channel 3.

FIG. 2 depicts the preferred embodiment of the medical apparatus for use in gynecological surgery and in particular laparoscopic assisted vaginal hysterectomy. The apparatus comprises an inflatable balloon cuff 15 which is capable of expanding to occlude the vagina 7. Inflation of the balloon is achieved by the addition of a gas or liquid, but preferably air, through a port 12 and the handle end 4 of the apparatus. The inflation gas or liquid is then transmitted to the balloon 15 through a channel 14. To maintain inflation and prevent uncontrolled deflation of the balloon the device may incorporate a restricting device 13. This may take the form of a valve, restriction or preferably a clamp.

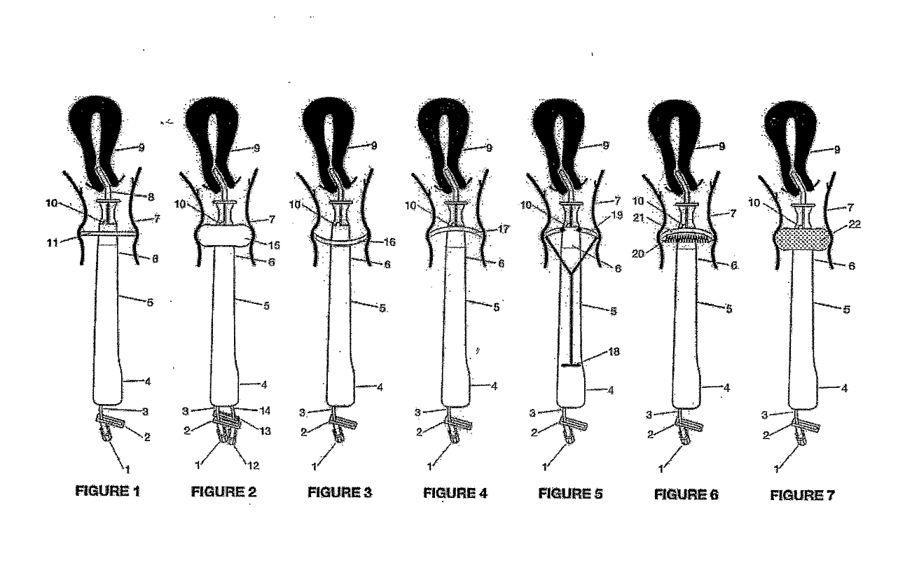

In another embodiment of the device as shown in FIG. 3, the diaphragm 16 is deformable and/or expandable and/or collapsible and is contoured such that the majority of it, or all of it, is dished towards the distal end of the apparatus.

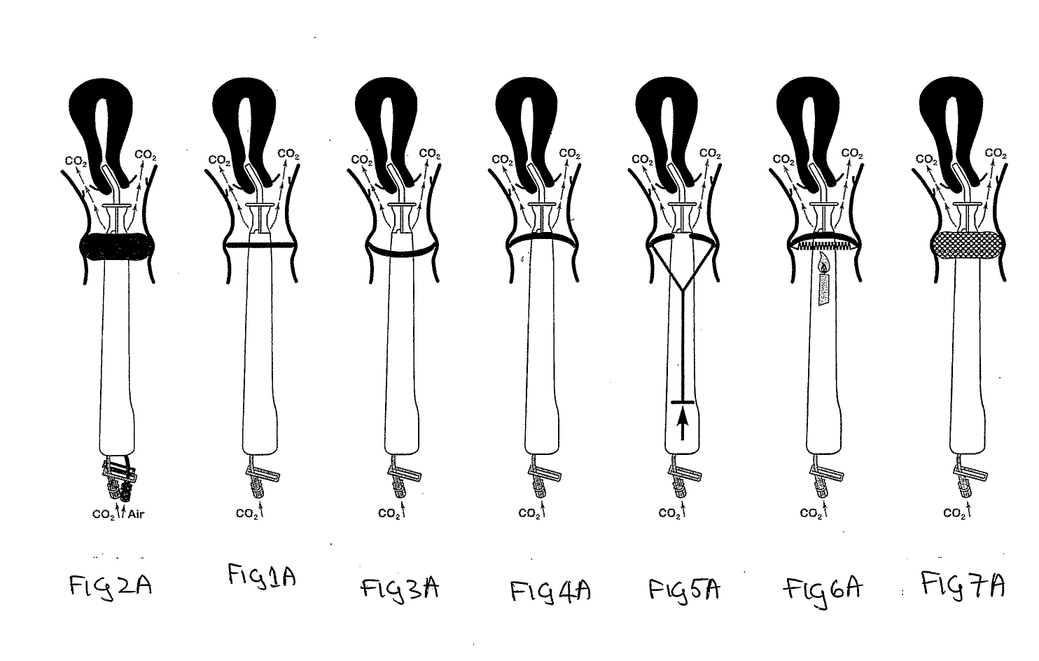

In another embodiment of the device as shown in FIG. 4, the diaphragm 17 is deformable and/or expandable and/or collapsible and is contoured such that the majority of it, or all of it, is dished away from the distal end of the apparatus.

In another embodiment of the device as shown in FIG. 5, the diaphragm 19 is configured such that it can be manually deformed and/or expanded and/or collapsed to occlude the vagina 7. The means of manually deploying the diaphragm 19 may be by the application of reciprocal and/or rotary motions by the operator on a control surface 18 situated at the handle end of the apparatus.

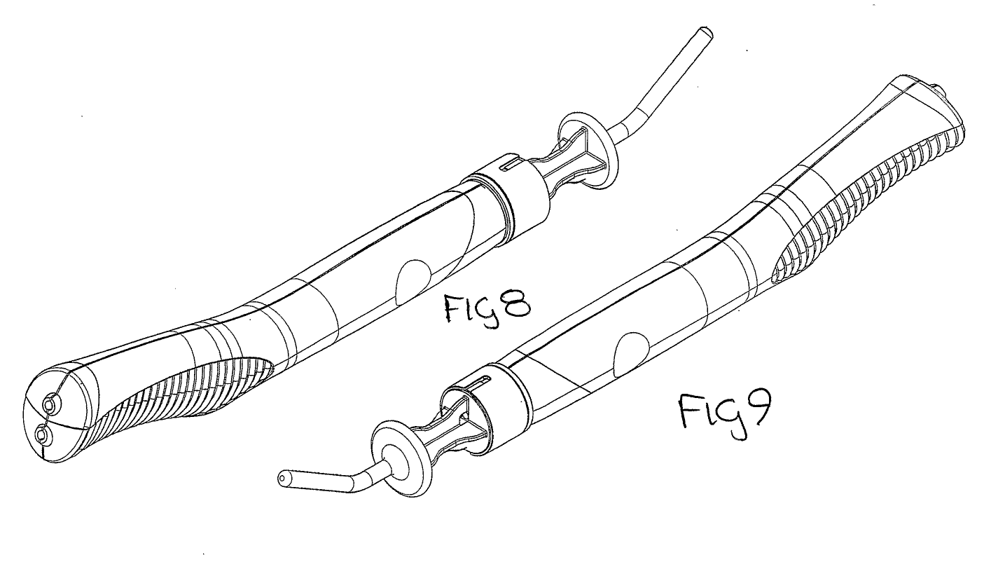

In another embodiment of the device as shown in FIG. 6, the diaphragm 21 may be configured such that it acts to occlude the vagina 7 in the presence or of absence of beat energy. This may be achieved through the addition of a heat sensitive metallic spring 20 or mechanism such as a bimetallic compound or a shape memory material.

In another embodiment of the device as shown in FIG. 7, the diaphragm 22 may be configured to be self deploying, this may be realised through the use of a compressed material which when released deforms and/or expands and/or collapses to occlude the vagina 7.

FIGS. 1A through 7A show gas flows of the respective devices of FIGS. 1 to FIG. 7. In each instance depending on the arrangement there is in use a ducting of a gas (e.g. carbon dioxide) from the manipulated end via appropriate tubing to issue out in the space beyond the diaphragm or inflated bladder. Also shown in the instance of FIG. 2A is the inflation flow of a gas, for example, of air to the inflatable bladder.

In the construction of the device as shown in FIGS. 8 to 10 preferably there are five major components which lock together either by means of an adhesive, mechanical fastening or (ultrasonic) welding, or any mixture of such means, to form the completed device. Any suitable materials can be utilised for the components of the device of FIGS. 8 to 10 but preferably they are of a suitable sterilisable material. A suitable plastics or metal can be used for each, e.g. structural component (e.g. medically certified plastics such as ABS, styrenes, etc. or starch based materials, etc or a metal e.g. SS, or other appropriate metal).

The diaphragm is preferably a silicone, latex or other similar medically certified elastic material. A thermally activated diaphragm can be of nitinol or other shape memory alloy.

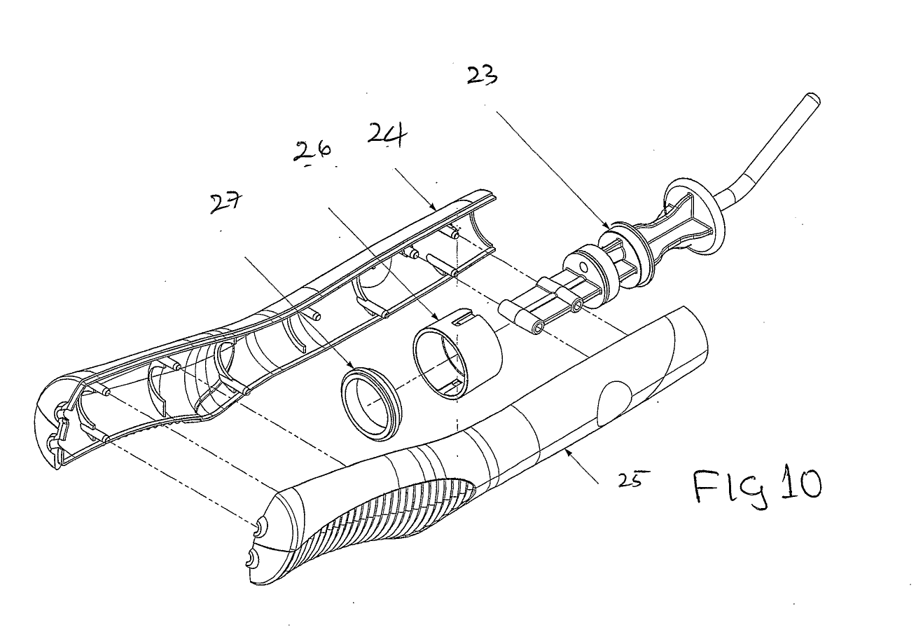

Shown specifically in FIG. 10 is a tip 23, a left body moulding 24, a right body moulding 25, a sleeve 25 and a compression ring 27.

It can be seen a he left and right body moulding 24 and 25 can be mated reliant on pins of one component fixing into appropriate receiving parts of the other and vice versa. Desirably a compression ring 27 acts on the sleeve 25 to hold the left and right moulding parts together in addition to whatever fixing of one to the other is otherwise provided.

Also it can be seen that the double porting from the manipulation end for two ducting tubes an inflatable diaphragm as in FIG. 1 (as in FIG. 2) to be fitted as an option for it.

In the preferred arrangement as shown in FIG. 10 the tip 23 is an component with a major portion thereof captured between the mouldings 24 and 25 with pins of each of the mouldings 24 and 25 being received within sleeves of the moulded tip component 23 on the centre lines shown. The tip has the requisite manipulator end suitable for the purpose previously described.

It is desirable therefore that the tip 23 as a separate component be located firmly between the moulded parts.

A pull on cuff (deformable but resilient) is then pulled over the tip onto the device at or near the sleeve 25. Gas tightness is to be ensured. Less critical is the fit if an inflatable cuff is pulled on provided it can connect to an inflating conduit.

Another embodiment is a device where the handle and tip are one piece with an integral cylindrical section between them. Rather than being injection moulded (as is preferred for the embodiment of FIGS. 8 to 10), the device may be thermoformed. Thermoforming (twin sheet) would result in a device that has a wall thickness (hollow) and require no assembly other than stretching the deformable but resilient cuff (dished away or dished towards the distal end) (- as in FIGS. 3 and 4 respectively) over the tip on to the cylindrical section near the tip to complete the assembly process.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

1-10. (canceled)

11. Surgical apparatus suitable for enabling establishment of a pneumoperitoneum and for uterus manipulation during a vaginal hysterectomy procedure, the apparatus comprising or including:
an elongate housing assembly having a handle end,
a manipulator tip component or assembly captured in part by said housing assembly distally of said handle end thereof thereby to define, beyond the housing assembly, a manipulator tip, and
a diaphragm about the housing assembly and/or manipulator tip component or assembly;
wherein there is defined at least one passageway for passing gas from the handle end of the housing assembly to at least one exit (exit(s)) in or adjacent to the manipulator member or assembly but beyond said diaphragm;
and wherein the diaphragm can be positioned in the vaginal tract and either without further deployment, or with further deployment (ie, inflation or other activation), establish a sufficient seal to define a gas inflatable peritoneal and abdominal cavity in which a pneumoperitoneum can be established by the passage of gas in said passageway and its egress from said one or more exit(s);
and wherein the manipulator tip can be manipulated sufficiently by the handle end of the housing assembly, and without movement of the manipulator tip relative to said housing assembly, despite the gas sealing presence of the diaphragm to allow manipulation of the uterus.

12. The apparatus according to claim 11, wherein the diaphragm comprises a flat ring of fixed volume.

13. The apparatus according to claim 11, wherein the diaphragm comprises a ring of fixed volume, the majority of which, or all of which is, dished towards the distal end of the apparatus.

14. The apparatus according to claim 11, wherein the diaphragm comprises a ring of fixed volume, the majority of which, or all of which is, dished away from the distal end of the apparatus.

15. The apparatus according to claim 11, wherein the diaphragm may be mechanically deployed from a collapsed or relaxed state to an expanded less relaxed state.

16. The apparatus according to claim 11, wherein the diaphragm may be thermally deployed from a collapsed state to an expanded state.

17. The apparatus according to claim 11, wherein the diaphragm may be self deploying from a collapsed state to an expanded state.

18. The apparatus according to claim 11, wherein the at least one passageway for passing gas to the distal end of the device has multiple exit points.

19. The apparatus according to claim 11, wherein said diaphragm is immediately about the housing assembly.

20. A method of laparoscopic assisted vaginal hysterectomy comprising or including the steps of:
(i) inserting apparatus comprising or including
an elongate housing, having a handle end and a manipulator end;
a diaphragm situated distal the handle end of the housing;
a manipulator tip component or assembly held by the elongate housing in the manipulator end and extending a manipulator tip away from said housing, and
a passage for passing gas from the handle end of the housing, to at least one exit from the housing in or adjacent to the manipulator and distal of the handle end beyond the diaphragm,
into a vagina, and if necessary deploying the diaphragm, so as to have the diaphragm fix the apparatus in place and to provide a sealed peritoneal and abdominal cavity;
(ii) passing a gas through said passage and into the peritoneal and abdominal cavity to create pneumoperitoneum;
(iii) utilising the manipulator tip to manipulate the uterus from within the uterus prior to vaginal cut; and
(iv) utilising the manipulator tip to manipulate the uterus from outside the uterus after vaginal cut.
